(19) United States
(12) Patent Application Publication
(10) Pub. No.: US 2008/0058955 A1
(43) Pub. Date: Mar. 6, 2008

(54) PROSTHETIC TESTICLE

(75) Inventors: Gary Bradford Shirley, Bloomington, IN (US); Ram H. Paul, Bloomington, IN (US); Brian C. Case, Lake Villa, IL (US)

Correspondence Address: BRINKS HOFER GILSON & LIONE/CHICAGO/COOK, PO BOX 10395, CHICAGO, IL, 60610, US

(73) Assignee: Vance Products Incorporated d/b/a Cook Urological Incorporated, Spencer, IN (US)

(21) Appl. No.: 11/844,198

(22) Filed: Aug. 23, 2007

Related U.S. Application Data
(60) Provisional application No. 60/840,910, filed on Aug. 29, 2006.

(51) Int. Cl. A61F 2/02; A46B 11/00

(52) U.S. Cl. 623/23.72; 401/118; 623/23.75

(57) ABSTRACT

A prosthetic testicle is provided that is able to mimic the movement and feel of a natural testicle while at the same time being able to remodel itself into native tissue. Additionally, the prosthetic testicle is able to provide long-term delivery of drugs into the patient.

### RELATED APPLICATION

The present patent document claims the benefit of the filing date under 35 U.S.C. §119(e) of Provisional U.S. Patent Application Ser. No. 60/840,910, filed Aug. 29, 2006, which is hereby incorporated by reference in its entirety.

### FIELD OF THE INVENTION

The invention relates to medical prosthetic devices and, in particular, to prosthetic devices implantable in the scrotum.

### BACKGROUND OF THE INVENTION

The testes of male mammals are responsible for the creation and circulation of a family of male sex hormones called androgens. The family of androgen hormones includes, among others, dihydrotestosterone, rostenedione, and testosterone. Male testosterone levels are lowered when one or both testicles are removed or lost as a result of trauma, birth defect, or disease. Lowered testosterone levels may negatively impact a male's genitalia growth, skeletal muscle development, skin condition, bone development, beard growth, body hair growth, and deepening of the voice. Testosterone loss also may adversely affect blood cells, blood lipids, erectile function, and the body's ability to process insulin.

Besides physical effects, lowered testosterone levels due to the loss of one or both testicles may also cause psychological problems, including but not limited to, feelings of shame, embarrassment, inadequacy, and loss of libido.

A patient with sufficiently lowered testosterone levels may undergo testosterone replacement therapy. There are a number of testosterone replacement preparations available today, including oral preparations, patches, gels, and injections. Each of these, however, suffers from adverse side effects.

Oral preparations in the United States must have the ability to be taken by mouth in an inert state, to be absorbed within the gastrointestinal tract, and to be activated by the liver. This may lead to liver dysfunction. Additionally, oral preparations provide for minimal testosterone delivery-time and thus, require multiple doses per day.

Testosterone patches, including both transcrotal patches and torso patches, also suffer from negative side effects. Transcrotal patches are placed onto the scrotum because the scrotal-skin provides for good testosterone absorption. However, the use of patches requires shaving of the area and even sometimes the use of a hairdryer to help the patch adhere. Patches also suffer from adverse side effects that include dermatitis, lesions, and skin rashes to the area where the patch is placed.

Testosterone gels are becoming more popular. However, they, too, suffer adverse consequences including limited drug delivery effectiveness. As a result, the patient is required to reapply the gel often.

Testosterone injections are also common. Side effects from injections include a super-physiologic testosterone level during the first few days after receiving the injection. Thereafter, the testosterone levels drop and are virtually non-existent by the time the patient is due for another injection. Thus, injections result in an undesired peak-and-valley effect. Furthermore, injections often have to be administered bi-weekly or monthly causing the patient to endure inconvenient trips to their physician's office as well as the pain and discomfort that result from each injection.

Tissue-engineered testicular prostheses comprising chrondrocytes and/or Leydig cells, described by U.S. Pat. No. 6,620,203 and published U.S. Patent Application No. US2002/0091448A1, similarly provide undesirable rapid increases in testosterone levels upon implantation, including a brief period of high super-physiologic testosterone levels within less than one day after implantation, followed by an undesirable rapid decline in testosterone levels.

Silicone or saline-filled prosthetic testicle described in U.S. Pat. Nos. 6,060,639 and 5,653,757 also suffer from adverse side effects, including that the prosthetic testicle be filled with a fluid before being used in a patient. Thus, the prosthetic testicle may be overfilled causing the device to crack and leak fluid into the patient. Additionally, as a result of trauma to the scrotum, the prosthetic testicle may crack while implanted within the patient and leak fluid. Fluid leakage into the patient can cause adverse side effects such as infection.

Silicone testicles suffer from other negativities besides adverse health effects. For example, such prosthetic testicles can be uncomfortable for the patient because they are hard, rigid, and un-natural feeling. Additionally, the inability to mimic a natural testicle structure may cause the prosthetic testicle to sit abnormally within the scrotum causing additional pain, stretching, and deformation. Furthermore, prosthetic testicles are unable to provide long-term delivery of testosterone and other drugs.

What is needed is a prosthetic testicle that is comfortable for the patient and has a lowered risk of infection. Further needed is a prosthetic testicle that is able to provide long-term drug, namely testosterone, delivery to a patient.

### BRIEF SUMMARY OF THE INVENTION

A first aspect of the invention includes a prosthetic testicle having an interior having a testicular-shape and a porous coating covering the interior.

A second aspect of the invention includes a prosthetic testicle having a reservoir configured for storing and releasing at least one drug. The reservoir is surrounded by an interior having a testicular-shape. The interior comprises at least one biodegradable material, and the interior is surrounded by a porous coating.

Additionally, a method for refilling a prosthetic testicle is provided. The method includes providing a prosthetic testicle having a reservoir configured for storing and releasing at least one drug. The reservoir is surrounded by an interior having a testicular-shape. The interior comprises at least one biodegradable material, and the interior is surrounded by a porous coating. The method further includes removing a residual drug from the reservoir, rinsing the reservoir with a fluid, and injecting a suitable amount of drug into the reservoir.

### BRIEF DESCRIPTION OF THE SEVERAL VIEWS OF THE DRAWINGS

The embodiments will be further described in connection with the attached drawing figures. It is intended that the drawings included as a part of this specification be illustrative of the embodiments and should in no way be considered as a limitation on the scope of the invention.

FIG. 1 is a cross-sectional view of a prosthetic testicle;

FIG. 2 is a magnified side view of a prosthetic testicle;

FIG. 3 is a cross-sectional view of an alternate embodiment of a prosthetic testicle; and

FIG. 4 is a side view of an alternate embodiment of a prosthetic testicle.

### DETAILED DESCRIPTION OF PRESENTLY PREFERRED EMBODIMENTS

The exemplary embodiments disclosed herein provide an apparatus that is suitable for implantation into the scrotum that can provide long-term drug delivery to the patient. The patient may include both human and veterinary patients.

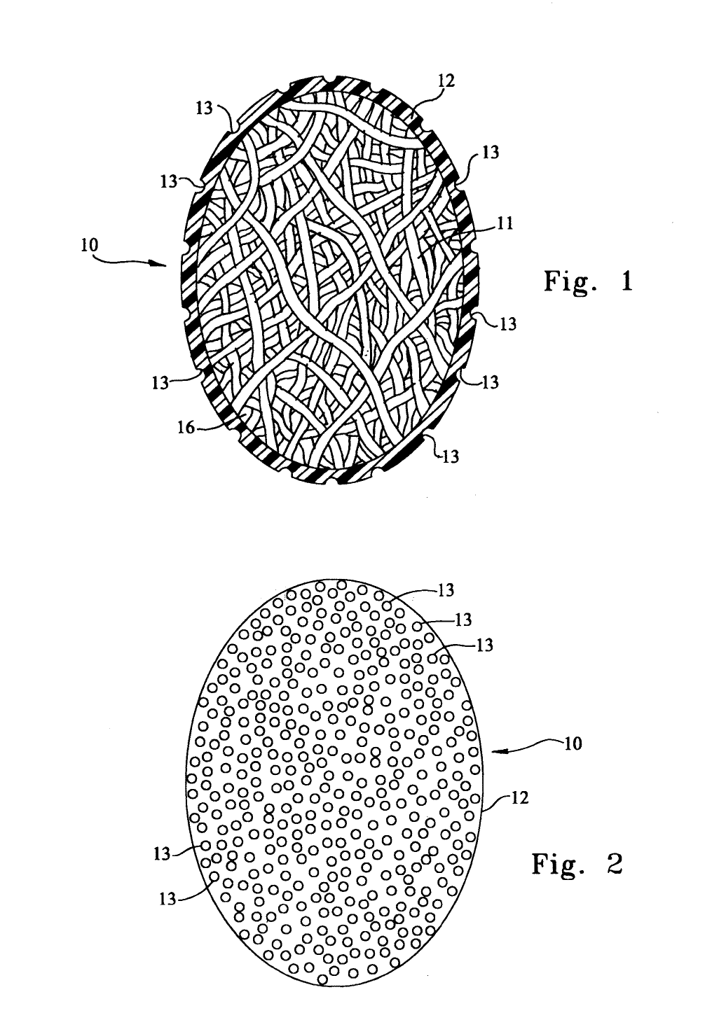

A more detailed description of the embodiments will now be given with reference to FIGS. 1-4. Throughout the disclosure, like reference numerals and letters refer to like elements. The present invention is not limited to the embodiments illustrated; to the contrary, the present invention specifically contemplates other embodiments not illustrated but intended to be included in the claims.

FIG. 1 is a cross-sectional view of an illustrative embodiment of a prosthetic testicle 10. Interior 11 that is configured to have the shape of a testicle comprises a suitable collagenous material, the fibers of which are preferably spun into a testicular-shape. Interior 11 is not limited to being formed via fibers spun into a testicular-shape; additionally, interior 11 can be made from a plurality of suitable material sheets that are rolled into a testicular-shape; as well as from suitable material foam formed into a testicular-shape.

Suitable collagenous materials include, but are not limited to: purified or reconstituted collagen; bovine or other mammalian pericardium; decellularized dermis; submucosa tissue such as urinary bladder submucosa, stomach submucosa, small intestine submucosa, and uterine submucosa; serosa tissue such as bovine serosa; basement membrane tissue such as liver basement membrane; autologous, allogenic, or xenogenic fascia lata; and so on. Materials which constitute a collagen-based extracellular matrix (ECM) are preferred. In general, mammalian tela submucosa tissues, which are collagen-based and thus predominantly collagen, are preferred ECM materials. These tissues may be procured from the alimentary, respiratory, urinary, or genital tracts of animals. Particularly suitable collagenous materials include ECMs such as submucosa, renal capsule membrane, dermal collagen, dura mater, pericardium, fascia lata, serosa, peritoneum or basement membrane layers, including liver basement membrane. Suitable submucosa materials for these purposes include, for instance, intestinal submucosa, including small intestinal submucosa, stomach submucosa, urinary bladder submucosa, and uterine submucosa. Submucosa or other ECM tissue used in the invention is preferably highly purified, for example, as described in U.S. Pat. No. 6,206,931 to Cook et al., incorporated herein by reference in its entirety.

A preferred material is small intestine submucosa (SIS) obtained from a porcine source, although the material for interior 11 of prosthetic testicle 10 is not limited to SIS. Preferably, interior 11 includes an SIS material derived from porcine tela submucosa that is disinfected prior to delamination by the preparation disclosed in U.S. Patent Application Publication No. US2004/0180042A1 by Cook et al., published Sep. 16, 2004 and incorporated herein by reference in its entirety. Most preferably, the tunica submucosa of porcine small intestine is processed in this manner to obtain the ECM material. This method is believed to substantially preserve the aseptic state of the tela submucosa layer, particularly if the delamination process occurs under sterile conditions. Specifically, disinfecting the tela submucosa source, followed by removal of a purified matrix including the tela submucosa (e.g. by delaminating the tela submucosa from the tunica muscularis and the tunica mucosa), may minimize the exposure of the tela submucosa to bacteria and other contaminants. In turn, this enables minimizing exposure of the isolated tela submucosa matrix to disinfectants or sterilants if desired, thus substantially preserving the inherent biochemistry of the tela submucosa and many of the tela submucosa's beneficial effects.

Optionally, the ECM materials within interior 11 may be cross-linked by any suitable method. Cross-linked materials tend to be less bioresorbable than non-cross linked materials. Cross-linking agents can be used to form cross-linking regions within interior 11. Cross-linking can be provided by chemical and/or light-induced treatment of the material forming interior 11. Chemical cross-linking can also be used to join layers of material together. In a first aspect, a portion of interior 11 can be cross-linked by contacting interior 11 material with a chemical cross-linking agent comprising glutaraldehyde, albumin, formaldehyde or a combination thereof. Other chemical cross-linking agents include epoxides, epoxyamines, diimides, and other difunctional/polyfunctional aldehydes. Cross-linking agents comprising aldehyde functional groups may be highly reactive with amine groups in proteins, such as collagen. Cross-linking agents may also include epoxyamines, which include both an amine moiety (e.g. a primary, secondary, tertiary, or quaternary amine) and an epoxide moiety. For example, an epoxyamine cross-linking agent can be a monoepoxyamine compound or a polyepoxyamine compound. Glutaraldehyde and polyepoxides are particularly preferred cross-linking agents for ECM materials. Alternatively, the material can be subjected to a form of energy to introduce cross-linking. For example, energy treatment suitable for use in the invention includes exposing the material to ultraviolet light, heat, or both. Cross-linking of natural polymers or synthetic polymers can also be accomplished with lyophilization, adhesives, pressure and or/heat.

In general, the process to form cross-linked material is conducted for a suitable amount of time. For example, the cross-linking agent may be allowed to penetrate through the material. Also, the cross-linking process generally reaches a point of completion at which time the properties of the material are essentially stable with respect to any additional measurable changes upon further contact with the cross-linking agent. Presumably, at completion, many, if not all, of the available functional groups of the material for cross-linking have reacted with a cross-linking agent. Since the formation of a fully cross-linked material is a slow process, the degree of cross-linking of the material at the cross-linking region can be selected to range from very low levels of cross-linking to complete cross-linking.

Interior 11 may be seeded with cells or biomolecules such as growth factors. In one example, the cells or biomolecules may be harvested from a healthy section of the individual's tissue, expanded in vitro using culture techniques, and seeded onto interior 11. In another example, chondrocytes for seeding into interior 11 can be obtained from other donor's tissues or from existing cell lines. Utilizing stem cell technology, mesenchymal cells obtained from bone marrow can also be differentiated into chondrocytes under appropriate culture conditions as described by, e. g., Butnariu-Ephrat et al., Clinical Orthopaedics and Related Research, 330:234-243, 1996. Interior 11 may also be seeded or doped with any other biomolecule or bioactive. ECM materials, when used, may naturally retain growth factors or other bioactive components native to the source tissue. For example, the submucosa tissue may include one or more growth factors such as basic fibroblast growth factor (FGF-2), transforming growth factor beta (TGF-beta), epidermal growth factor (EGF), and/or platelet derived growth factor (PDGF). As well, submucosa tissue used in the invention may include other biological materials such as heparin, heparin sulfate, hyaluronic acid, fibronectin and the like.

Interior 11 of prosthetic testicle 10 may also be formed from a tissue engineered product involving in vitro cell culture techniques, such as the use of stem cells or other cells in combination with SIS or other biodegradable material. One such technique is to seed cells onto SIS material or other biodegradable scaffold in the shape of testicle 10 or interior 11. The term “biodegradable,” as used herein refers to materials which dissipate within the body by any mechanism, including enzymatic or chemical degradation. Other biodegradable scaffolds, some of which are mentioned elsewhere, include collagen, extra-cellular matrix materials (ECM) such as SIS, and synthetic polymers such as polyglycolides, polylactides, and their co-polymers. Interior 11 may also be formed using stem cells. One technique is to culture stem cells in a specific environment to induce cell differentiation. The newly derived cells or tissue, created from stem cells, could be formed directly into interior 11 or seeded onto a scaffold material to form interior 11 of the prosthetic testicle 10. Cell types used in this fashion include, but are not limited to, fibroblasts, smooth muscle cells, chondrocytes, and Leydig cells. The biodegradable scaffold material may be selected from any suitable biocompatible biodegradable polymer having the desired physical properties of resilience and absorptivity. Desirably, the biodegradable material includes polylactic acid (poly lactide) (PLA), polyglycolic acid (poly glycolide) (PGA), polylactic glycolic acid (poly lactide-co-glycolide) (PLGA), poly-4-hydroxybutyrate, poly-L-lactide (PLLA), polydioxanone, polygluconate, polylactic acid-polyethylene oxide copolymers, poly(hydroxybutyrate), polyanhydride, polyphosphoester, poly(caprolactone), polycarbonates, polyamides, polyanhydrides, polyamino acids, polyortho esters, polyacetals, polycyanoacrylates and degradable polyurethanes. The biodegradable scaffold material could also be an ECM material, such as SIS.

Suitable collagenous material, and in particular SIS obtained from a porcine source, provides many advantages to the prior art's use of silicone or saline-filled prosthetic testicles. First, SIS provides no risk of leakage or deflation. Because prosthetic testicle 10 is not filled with any fluid, there is less risk that the device will crack and require replacement due to overfilling or from scrotal trauma. Second, compared to silicone, SIS provides little risk of infection. An additional advantage is that natural SIS material can remodel overtime into native tissue. The terms “remodel” or “remodelable” as used herein refer to the ability of a material to allow or induce host tissue growth, proliferation or regeneration following implantation of the material in vivo. Remodeling can occur in various microenvironments within a body, including without limitation soft tissue, sphincter muscle region, tendon, ligament, bone tissues, and cardiovascular tissues. Upon implantation of a remodelable material, cellular infiltration and neovascularization are typically observed over a period of about five days to about six months or longer, as the remodelable material acts as a matrix for the ingrowth of adjacent tissue with site-specific structural and functional properties. The remodeling phenomenon which occurs in mammals following implantation of submucosal tissue includes rapid neovascularization and early mononuclear cell accumulation. Mesenchymal and epithelial cell proliferation and differentiation are typically observed by one week after in vivo implantation and extensive deposition of new extracellular matrix occurs almost immediately.

The exterior of prosthetic testicle 10 is covered with a porous outer shell coating 12 (depicted in FIGS. 1 and 2) to prevent interior 11 from growing larger than its implanted size and to prevent interior 11 from attaching to the scrotal wall. Coating 12 is applied to the exterior of interior 11 having a thickness range of about 0.001-0.1 inches; the preferred thickness being 0.01-0.05 inches because such thickness will retain the shape of interior 11 and allow, in combination with pores 13, drugs to elute therefrom.

Interior 11 is preferably dip-coated with coating 12, preferably a polyurethane, to achieve an even coat. Other methods of coating application are contemplated, including, but not limited to, curing the coating into a thin film and rolling coating 12 onto interior 11, spraying coating 12 onto interior 11, as well as forming the outer shell coating by vacuum forming, compression molding, and injection molding.

Outer shell coating 12 comprises a biocompatible polyurethane coating. One preferred biocompatible polyurethane coating is a polyurethaneurea sold under the tradename THORALON, developed by Thoratec (Pleasanton, Calif.). Thoralon is a preferred coating due to the fact that it is highly biocompatible, strong, flexible, and can be slick or lubricious in certain forms (e.g., thin films). Descriptions of suitable biocompatible polyurethaneureas are described in U.S. Patent Application Publication No. 2002/0065552 A1 and U.S. Pat. No. 4,675,361, both of which are incorporated herein by reference. Briefly, these publications describe a polyurethane base polymer (referred to as BPS-215) blended with a siloxane containing surface modifying additive (referred to as SMA-300). The SMA-300 component (THORATEC) is a polyurethane comprising polydimethylsiloxane as a soft segment and the reaction product of 4,4′-diphenylmethane diisocyanate (MDI) and 1,4-butanediol as a hard segment. A process for synthesizing SMA-300 is described, for example, in U.S. Pat. Nos. 4,861,830 and 4,675,361, which are incorporated herein by reference. The BPS-215 component (THORATEC) is a segmented polyetherurethane urea containing a soft segment and a hard segment. The soft segment is made of polytetramethylene oxide (PTMO), and the hard segment is made from the reaction of 4,4′-diphenylmethane diisocyanate (MDI) and ethylene diamine (ED). The concentration of the surface modifying additive may be in the range of 0.5% to 5% by weight of the base polymer.

A biocompatible polyurethane coating will help prosthetic testicle 10 maintain a more natural-testicle feel and will help prosthetic testicle 10 to move about naturally within the scrotal sack. However, coating 12 is not limited to Thoralon; other biocompatible polymers are also contemplated. The polymer coating can also be a polytetrafluoroethylene (PTFE) coating. Polymer coating can also comprise a hydrophilic polymer selected from the group comprising polyacrylate, copolymers comprising acrylic acid, polymethacrylate, polyacrylamide, poly(vinyl alcohol), poly(ethylene oxide), poly(ethylene imine), carboxymethylcellulose, methylcellulose, poly(acrylamide sulphonic acid), polyacrylonitrile, poly(vinyl pyrrolidone), agar, dextran, dextrin, carrageenan, xanthan, and guar. The hydrophilic polymers can also include ionizable groups such as acid groups, e.g., carboxylic, sulphonic or nitric groups. The hydrophilic polymers may be cross-linked through a suitable cross-binding compound. The cross-binder actually used depends on the polymer system: if the polymer system is polymerized as a free radical polymerization, a preferred cross-binder comprises two or three unsaturated double bonds.

Coating 12 can also be loaded with a variety of bioactives. Coating 12 is capable of releasing the bioactive into the body at a predetermined time and at a predetermined rate. Such polymeric coatings include drug-eluting matrix materials described in U.S. Pat. Nos. 5,380,299, 6,530,951, 6,774,278 and U.S. patent application Ser. Nos. 10/218,305, 10/223,415, 10/410,587, 10/000,659, and 10/618,977, all of which are incorporated in their entirety herein by reference. Alternatively, different drug-eluting polymer coatings can be coated onto interior 11 as well. Coating 12 can include any bioactive commonly known to those skilled in the art to help reduce tissue irritation incurred as a result of prosthetic testicle 10 being in contact with tissue for a prolonged period of time.

Accordingly, coating 12 of the prosthetic testicle 10 may include testosterone and/or one or more drugs to resist infection or rejection of prosthetic testicle 10. The drugs may include rifampin and minocycline, or other antibiotic/antimicrobial drugs. These drugs may include, but are not limited to, a mixture of rifampin and minocycline, a non-steroidal anti-inflammatory drug (NSAID) (including, but not limited to, aspirin, salsalate, diflunisal, ibuprofen, ketoprofen, nabumetone, piroxicam, naproxen, diclofenac, indomethacin, sulindac, tolmetin, etodolac, ketorolac, oxaprozin, and celecoxib), a penicillin, a cephalosporin, a carbepenem, a beta-lactam, an antibiotic, a macrolide, a lincosamide, an aminoglycoside, a glycopeptide, a tetracyline, a chloramphenicol, a quinolone, a fucidin, a sulfonamide, a trimethoprim, a rifamycin, an oxaline, a streptogramin, a lipopeptide, a ketolide, a polyene, an azole, an echinocandin, alpha-terpineol, methylisothiazolone, cetylpyridinium chloride, chloroxyleneol, hexachlorophene, chlorhexidine and other cationic biguanides, methylene chloride, iodine and iodophores, triclosan, taurinamides, nitrofurantoin, methenamine, aldehydes, azylic acid, rifampycin, silver, benzyl peroxide, alcohols, carboxylic acids and salts, and silver sulfadiazine. Other examples of suitable antibiotics include amoxicillin, trimethoprim-sulfamethoxazole, azithromycin, clarithromycin, amoxicillin-clavulanate, cefprozil, cefuroxime, cefpodoxime and cefdinir. Anti-rejection drugs help to prevent rejection of the transplant by the body. Anti-rejection drugs may include, but are not limited to, neomycin, cyclosporine, prednisone, and tacrolimus.

Pores 13 (also depicted in FIG. 2) contained within coating 12 are about 1-100 microns in size. The size of pores 13 is preferably about 10-30 microns so as to allow nutrients and single cells to infiltrate interior 11 and encourage interior 11 to remodel into native tissue, thus, avoiding degradation of interior 11. Pores 13, having the size of about 10-30 microns, also reduce the ability of any portion of interior 11 to exit through pores 13 and attach to the scrotal wall. The preferred degree of porosity may be expressed as a void-to-volume ratio of about 25%-75%, or greater; however, other degrees of porosity are contemplated.

Interior 11 can also be loaded with testosterone 16, or other drugs for hormone replacement therapy, as well as one or more drugs to resist infection or rejection. However, depending on the patient's needs, interior 11 need not be loaded with any drugs. A typical male patient, ages 17 to 65, releases 5-6 mg of testosterone per day, with plasma testosterone levels maintained at about 3-10 mg/ml. However, it is often times necessary to release more testosterone than the typical body creates, because some of the testosterone therapy is absorbed into the bodily tissue before reaching systemic circulation; additionally, some testosterone may be rendered inert through chemical reactions that occur within the body. Therefore, it is preferred that interior 11 be loaded with about 25 mg of testosterone for each day prosthetic testicle 10 is to remain within the body without needing to be refilled. For example, a 28 day supply of testosterone released as 25 mg per day would require about 0.7 grams of testosterone to be loaded into interior 11. Additionally, a 3-month supply would require about 2.1 grams of testosterone to be loaded into interior 11; whereas a 1-year supply would require about 8.4 grams of testosterone to be loaded into interior 11. Thus, interior 11 is preferably loaded with about 0.1-10 grams of testosterone or other drugs. However, greater or lesser amounts are contemplated based upon the needs of the patient, the porosity of interior 11, and the porosity of coating 12.

Interior 11 may be loaded with penicillin by dipping interior 11 in a suitable liquid medium containing HAMM's F12 medium (Gibco, New York, N.Y.) containing 10% fetal bovine serum with L-glutamine (292 μg/ml), penicillin (100 μg/ml) and ascorbic acid (50 μg/ml). Other media may also be used. For example, “standard cell growth media” may include Dulbecco's Modified Eagles Medium, low glucose (DMEN), with 110 mg/L pyruvate and glutamine, supplemented with 10-20% Fetal Bovine Serum (FBS) or 10-20% calf serum (CS) and 100 U/ml penicillin. Other standard media include Basal Medium Eagle, Minimal Essential Media, McCoy's 5A Medium, and the like, preferably supplemented as above (commercially available from, e.g., JRH Biosciences, Lenexa, Kan.; GIBCO, BRL, Grand Island, N.Y.; Sigma Chemical Co., St. Louis, Mo.). Any other suitable method for doping interior 11 with drugs can also be used.

Methods for doping interior 11 include, but are not limited to, dipping or soaking interior 11 into a suitable drug solution. Additionally, interior 11 can be reloaded with additional drugs 16 should the need arise. To reload interior 11, the physician injects through the scrotal sack a sufficient amount of testosterone 16 or other drug directly into interior 11.

The size of prosthetic testicle 10 is preferably 5-30 cm³ in order to replicate the size of a natural human testicle; however, other sizes are contemplated based upon the size and needs of the patient.

FIG. 3 depicts another illustrative embodiment of a prosthetic testicle 30. Prosthetic testicle includes an outer shell coating 12 as described previously. Additionally, interior 34 configured to have a testicular shape is made from suitable collagenous material foam formed into a testicular-shape having pores 13. However, interior 34 can also be made from a plurality of suitable material sheets that are rolled into a testicular-shape or from suitable material fibers spun into a testicular-shape. As described above, interior 34 can be loaded with one or more drugs to resist infection or rejection 16 as well as testosterone.

Additionally, prosthetic testicle 30 further includes a drug reservoir 33 having a porous shell 31 for holding and releasing drugs 35. Shell 31 is preferably made from a biocompatible polyurethane such as the polyurethaneurea sold under the tradename THORALON, or other suitable synthetic material, and is preferably about 0.05 inches thick. However, other thicknesses are contemplated, including but not limited to about 0.001-0.1 inches. Additionally, shell 31 can be made from a self-sealing silicone material, or other suitable self-sealing material, so that reservoir 33 can be later refilled while prosthetic testicle 30 remains resident in the patient. Shell 31 may comprise a non-biodegradable self-sealing elastomeric material. The term elastomeric as used herein refers to a substance which is capable of essentially rebounding to near its initial form or state after deformation. For example, shell 31 can comprise a self-sealing elastomeric material formed from a suitable polymer selected from the group consisting of polyurethane, polyurea, and polyurethaneurea.

Pores 36 contained within shell 31 have the size of about 1-30 microns. The size of pores 36 is preferably about 10-30 microns so as to allow drugs 35 to escape from reservoir 33 but reduce the ability of any portion of interior 34 to enter reservoir 33 through pores 36. The preferred degree of porosity is a void-to-volume ratio of about 25%-75%; however, other porosities are contemplated.

Reservoir 33 can be loaded with testosterone 35, or other drugs for hormone replacement therapy, as well as one or more drugs to resist infection or rejection. However, depending on the patient's needs, reservoir 33 need not be loaded with any drugs. A typical male patient, ages 17 to 65, releases 5-6 mg of testosterone per day, with plasma testosterone levels maintained at about 3-10 mg/ml. However, as described above, it is often times necessary to release more testosterone than the typical body creates, because some of the testosterone therapy is absorbed into the bodily tissue before reaching systemic circulation; additionally, some testosterone may be rendered inert through chemical reactions that occur within the body. Therefore, typically, it is preferred that reservoir 33 be loaded with about 25 mg of testosterone per day for the period of time in which prosthetic testicle 30 is to remain within the body without needing to be refilled; thus, reservoir 33 is preferably loaded with about 0.1-10 grams of testosterone or other drugs. However, greater or lesser amounts are contemplated based upon the needs of the patient, the porosity of interior 34, the porosity of outer coating 12, and the porosity of reservoir shell 31.

Interior 34 can be reloaded with additional drugs 16 should the need arise. To reload interior 34, the physician injects through the scrotal sack a sufficient amount of drugs 34 directly into interior 34.

Additionally, reservoir 33 can be reloaded with additional drugs 35, should the need arise. To reload reservoir 33, the physician injects a sufficient amount of testosterone 35, or other required drug, directly into reservoir 33. Additionally, before injecting drugs 35 into reservoir 33, the physician can drain any residual drugs 35 remaining within reservoir 33 and can also rinse (flush) the area with saline or other suitable fluid. Draining and flushing reservoir 33 prior to reloading it is beneficial for accurately determining the amount of drug to be reloaded into reservoir 33. Thus, the physician is better able to determine the amount of drug needing to be replaced when reservoir 33 is completely empty, rather than estimating an amount when some amount of drugs remains within reservoir 33.

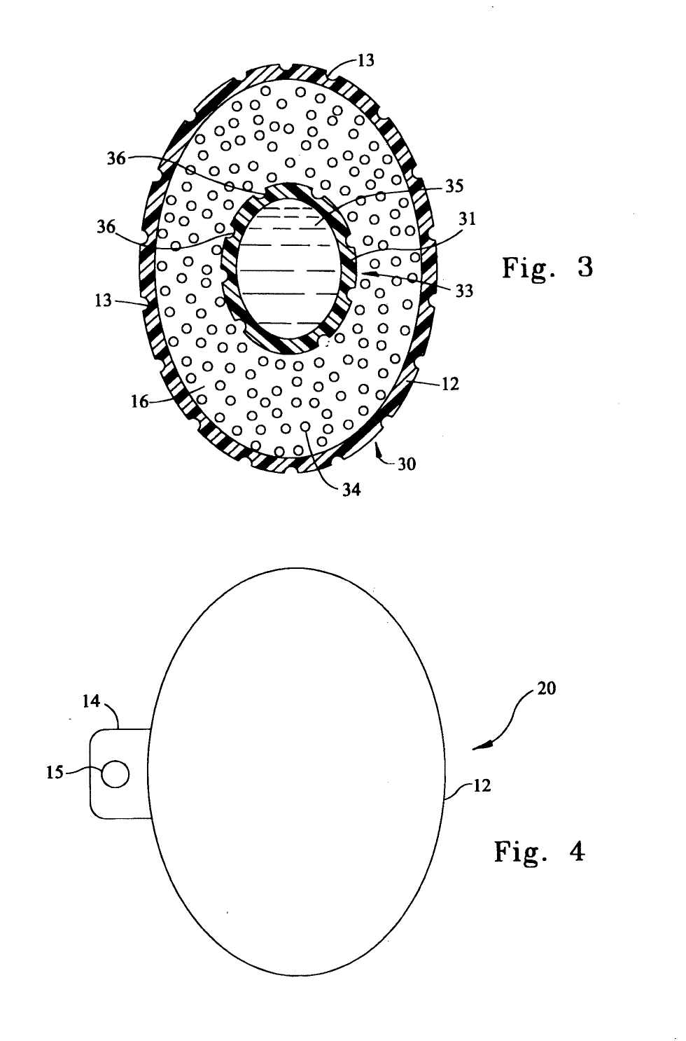

FIG. 4 depicts another illustrative embodiment of a prosthetic testicle 20 having a suture tab 14. Suture tab 14 is made from a biocompatible polyurethane; however the use of other biocompatible materials is contemplated. The polyurethaneurea sold under the tradename THORALON is a preferred material due to the fact that it is highly biocompatible, strong, and flexible. Suture tab 14 comprises a square-shape having the dimensions of about 25 mm²; larger and smaller dimensions are contemplated based on the size of prosthetic testicle 20 as well as the needs of the patient. Suture tab 14 further comprises a suture hole 15 where a suture (not shown) can be directed through so that prosthetic testicle 20 can be attached to the scrotal wall. It is preferred that if prosthetic testicle 20 is to be sutured into the scrotal wall, that such suture be done so that prosthetic testicle 20 is able to have some movement within the scrotal sack so as to prevent interior 11 from growing into the scrotal wall and to better simulate the feel of a natural testicle.

As is evident, the embodiments provide a very effective solution for replacement of a natural testicle using a more-natural feeling prosthetic testicle that is able to provide long-term drug release. The foregoing description and drawings are provided for illustrative purposes only and are not intended to limit the scope of the invention described herein or with regard to the details of its construction and manner of operation. It will be evident to one skilled in the art that modifications and variations may be made without departing from the spirit and scope of the invention. Changes in form and in the proportion of parts, as well as the substitution of equivalents, are contemplated as circumstances may suggest and render expedience; although specific terms have been employed, they are intended in a generic and descriptive sense only and not for the purpose of limiting the scope of the invention set forth in the following claims.

What is claimed is:

1. A prosthetic testicle comprising an interior having a testicular-shape and a porous coating covering the interior.

2. The prosthetic testicle of claim 1, wherein the interior comprises at least one biodegradable material.

3. The prosthetic testicle of claim 1, wherein the porous coating of the interior comprises a polymer.

4. The prosthetic testicle of claim 1, wherein at least one of the interior and the porous coating of the interior further comprises one or more drugs to resist infection or rejection.

5. The prosthetic testicle of claim 1, wherein the interior is loaded with a drug.

6. The prosthetic testicle of claim 5, wherein the drug is testosterone and prosthetic testicle is configured to release about 5-25 mg of testosterone per day.

7. The prosthetic testicle of claim 1, wherein the porous coating covering the interior has a porosity characterized by a void-to-volume ratio of about 25-75%.

8. The prosthetic testicle of claim 1, wherein the interior is configured for reloading with drugs.

9. The prosthetic testicle of claim 1, further comprising a reservoir configured for storing and releasing drugs, wherein the reservoir is disposed within the interior.

10. The prosthetic testicle of claim 9, wherein the reservoir further comprises a porous coating surrounding an exterior of the reservoir.

11. The prosthetic testicle of claim 9, wherein the porous coating of the reservoir has a porosity characterized by a void-to-volume ratio of about 25-75%.

12. The prosthetic testicle of claim 9, wherein the porous coating of the reservoir is self-sealing.

13. The prosthetic testicle of claim 9, wherein the reservoir is configured for loading with drugs.

14. A prosthetic testicle comprising:
a reservoir configured for storing and releasing at least one drug, wherein the reservoir is surrounded by an interior having a testicular-shape;
wherein the interior comprises at least one biodegradable material; and
wherein the interior is surrounded by a porous coating.

15. The prosthetic testicle of claim 14, wherein the prosthetic testicle is configured to release about 5-25 mg of testosterone per day.

16. The prosthetic testicle of claim 14, wherein an exterior of the reservoir further comprises a self-sealing polymer.

17. The prosthetic testicle of claim 14, wherein at least one of the porous coating or the interior further comprises one or more drugs to resist infection or rejection.

18. The prosthetic testicle of claim 14, wherein the reservoir is configured for reloading with a drug.

19. The prosthetic testicle of claim 14, wherein the porous coating surrounding at least one of the reservoir or the interior further comprises a biocompatible polyurethane.

20. A method for refilling a prosthetic testicle, the method comprising:
providing a prosthetic testicle having a reservoir configured for storing and releasing at least one drug, wherein the reservoir is surrounded by an interior having a testicular-shape; wherein the interior comprises at least one biodegradable material; and wherein the interior is surrounded by a porous coating;
removing a residual drug from the reservoir;
rinsing the reservoir with a fluid; and
injecting a suitable amount of drug into the reservoir.
